(19) United States
(12) Patent Application Publication
(10) Pub. No.: US 2011/0015521 A1
(43) Pub. Date: Jan. 20, 2011

(54) Means of Tracking Movement of Bodies During Medical Treatment

(75) Inventor: Ivan Faul, Boulder, CO (US)

Correspondence Address: The Marbury Law Group, PLLC, 11800 SUNRISE VALLEY DRIVE, SUITE 1000, RESTON, VA, 20191, US

(73) Assignee: Boulder Innovation Group, Inc., Boulder, CO (US)

(21) Appl. No.: 12/889,596

(22) Filed: Sep. 24, 2010

Related U.S. Application Data
(63) Continuation-in-part of application No. 12/818,850, filed on Jun. 18, 2010.
Application No. 12/818,850 is a continuation of application No. 10/808,459, filed on Mar. 25, 2004, now Pat. No. 7,742,804.
(60) Provisional application No. 60/457,567, filed on Mar. 27, 2003.

(51) Int. Cl. A61B 6/03; A61N 5/10; H04B 5/00

(52) U.S. Cl. 600/426; 250/492.1; 455/41.1

(57) ABSTRACT

Methods and systems enable accurate control of robotic treatments of internal features of a body by tracking movements of the exterior of the body. Such tracking enables programmed and automated or semi-automated surgical operations to compensate for movement of the patient's body during surgery. A tracking system that includes a marker, which may be disposable, that is attached to the body and accurately tracked in a three-dimensional coordinate system by a tracking system. Compensation for body movements may be accomplished by adjusting the movement or position of a surgical instrument, a surgical robot, a radiation source collimator and/or the operating room table. The markers may include a radiofrequency identifier (RFID) chip or memory chip that can be interrogated by the tracking system.

### RELATED APPLICATIONS

This application is a continuation in part of U.S. patent application Ser. No. 12/818,850 filed Jun. 18, 2010, which is a continuation of U.S. patent application Ser. No. 10/808,459 filed on Mar. 25, 2004 that issued as U.S. Pat. No. 7,742,804 on Jun. 22, 2010, which claims the benefit of priority to U.S. Provisional Application Ser. No. 60/457,567 filed on Mar. 27, 2003, the entire contents of all of which are incorporated herein by reference.

### FIELD

This invention is directed to the art of tracking the movement of objects, especially the involuntary movement of internal organs, or structural features, or the like, as a function of body movements caused by a patient's breathing or other voluntary or involuntary movement.

### BACKGROUND

It is widely known that internal tumors can succumb to radio surgery and that kidney stones can be broken up into gravel by impinging ultra sound energy on them. Tumors in the thoracic cavity or elsewhere in the body can be attacked by impinging laser, X-rays, or other high-energy radiation beams on them with sufficient power to kill the tumor cells. Similarly, stones accumulated in the kidney, gall bladder and the like can be treated with other radiation beams, such as ultra sound, in order to break up the stones into gravel that is small enough to pass out of the patient's system. It is obvious that, if the direction of the high-energy radiation beam is not exactly where it is supposed to be, even if it is off very slightly, the consequences are that the procedure is either ineffective or not completely effective. That is, for example, either that the entire tumor is not destroyed, or rendered necrotic, (because the high energy X-ray beam doesn't reach to the edge of the tumor) or normal, healthy tissue is destroyed, or rendered necrotic, (because the X-ray beam impinges on tissue outside the periphery of the tumor). Therefore, technicians go to extreme lengths to insure that the X-ray beam is properly focused exactly on the location of the tumor or other feature being treated.

It will be clear, however, that the patient being treated is breathing throughout the high-energy radiation treatment. Thus, the thoracic cavity (or other locations under treatment) is almost constantly moving as a function of normal breathing. Further, there is the risk that the patient will inadvertently sneeze or cough during treatment, which would have a sever impact on the accuracy of the impingement of the high-energy radiation. As the patient breathes, his chest moves and thus the alignment of the X-ray beam can move from being focused directly on the whole of the tumor, or other feature, to being off its target to a greater or lesser extent. The difference between on-target and slightly off-target need not be great. Even if the offset is very small, that difference can be critical to the success or failure of the treatment such as resection, or rendering the impinged tissue necrotic, or other treatment.

This situation is equally true for remote controlled and so called “surgeonless” operations that employ a solid scalpel rather than a radiation scalpel, such as a high energy X-ray beam. The scalpel is wielded by a remotely controlled machine that has been preprogrammed to follow a specific predetermined track or course, if the body being worked on moves during surgery, but the preprogrammed track has not been programmed to compensate for this movement, the scalpel will cut in the wrong place, at least part of the time. Also, when a remotely located surgeon is directly controlling the scalpel via remote-controlled means, and no preprogramming exists, real-time feedback of patient body motion is required to indicate to the remotely located surgeon, or to automated surgical equipment, that the patient or its organs have moved.

It is known in the surgical field that certain forms of surgery, particularly computer operated cranial image guided microneurosurgery, can be greatly assisted and improved by independently tracking the movements of a scalpel or probe while the functional ends of these instruments are out of line of sight of the surgeon. In this technique, these movements of the scalpel, or the like, are matched to the feature of the body that is being resected or rendered necrotic as it appears on a previously taken image of the portion of the intracranial tissue that is being resected or rendered necrotic (that is, the tumor). Thus, the probe or knife can be made to follow the contours of the diseased tissue as shown on a previously taken MRI, or the like, even where the surgeon cannot directly see the diseased tissue. Clearly it is very important that the patient's head or body be maintained absolutely still during the surgery, and this has been accomplished by severely clamping the head in suitable restraints prior to and during surgery. However, it is not always possible to maintain the cranium absolutely still during extended surgery.

It is also known (see U.S. Pat. No. 6,501,981 for example) to carry out treatments of internal features of a body while compensating for the inadvertent, or intentional, movement of the body during surgery. This reference discloses that this compensation is accomplished by periodically generating positional data about the internal target structure or feature that is being treated, continuously generating position data about the position of markers operatively associated with the body but positioned outside the body, and generating a correspondence between these sets of data.

As with most, if not all, medical instrumentalities, it is undesirable to employ an instrumentality with one patient that has been used by another patient. Further, it is important to use instrumentalities in connection with a patient that do not significantly adversely affect the treatment itself.

### SUMMARY

The various embodiments provide an apparatus for improving the accuracy of surgeonless treatment of internal features of a body of an animal, particularly a human, by tracking the movement of the body, which enables programmed and automated or semi-automated surgical operations to account for movement of the patient's body during surgery. Movement of at least a portion of the patient's body may be measured by a tracking system that includes a marker, which may be disposable, which is attached to the body and accurately tracked in a three-dimensional coordinate system by a tracking system. Such compensation may be accomplished by adjusting the movement or position of a surgical instrument, a surgical robot, a radiation source collimator or the operating room table. In an embodiment the markers or the harness connectable to the markers may include a circuit to enable it to automatically identify itself (e.g., in the form of a serial number) to the tracking system, such as a memory chip or radiofrequency identifier (RFID) chip or memory chip that can be interrogated by the tracking system.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIGS. 1A through 1D show an exploded view of the invention using a clip to connect the device with an external power source.

FIGS. 2A through 2D show an exploded view of the invention using a connector to join the device (emitter) with an external power source.

FIGS. 3A and 3B show an exploded view of an application of a tracking system of this invention to a patient through a film/fabric structure.

FIG. 4 is a perspective view of a remote LED housing and a plurality of optical fibers leading from that housing.

FIG. 5 is a perspective view of an LED attached to the end of an optical fiber.

FIG. 6 is a system block diagram of an automated operating room illustrating systems and components of the various embodiments.

FIG. 7 is a process flow diagram of an example method for conducting a surgery utilizing the various embodiments.

FIGS. 8A-8C show alternative configurations of embodiments employing RFID chips coupled to markers or to a remote LED housing.

FIG. 9 is a component block diagram of an example computer suitable for use with various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The word “exemplary” is used herein to mean “serving as an example, instance, or illustration.” Any implementation described herein as “exemplary” is not necessarily to be construed as preferred or advantageous over other implementations. Also, the term “X-ray” is used herein in a generic sense to encompass many different kinds of radiation beams, such as X-rays, gamma rays, laser beams, ultra sound, and other similar radiation scalpels/tools).

As used herein, the terms “marker” or “markers” refer to a device that can be placed on a patient to enable a tracker system to detect the position and movement of the patient within a coordinate system by tracking reflections or emissions of measurable radiation (e.g., light, UV light, IR light, sound, and magnetic fields). Since one or multiple markers may be used in a procedure references herein to “marker” and “markers” should be understood to encompass both a single marker and a plurality of markers as may be appropriate in a given implementation.

It is an object of this invention to provide an apparatus and system for improving the accuracy of surgeonless treatment of internal features of a body of an animal, particularly a human.

It is an additional object of this invention to provide methods and systems for modifying preprogrammed surgical operations (solid or radiation scalpel) to account for movement of the patient's body during surgery.

It is a further object of this invention to provide methods and systems, that are auxiliary to preprogrammed surgery, to cause the movement of the surgical equipment (e.g., surgical tool, radiation source, focusing mechanism of the radiation source, operating room table, and/or other operating tool) that is more accurate than has been possible by following the teachings of the prior art.

It is a still further object of this invention to provide disposable means for determining the movement of the patient and integrating such movement with preprogrammed treatment tracks.

Other and additional objects of this invention will become apparent from a consideration of this entire specification and claims.

In accord with and fulfilling these objects, one aspect of this invention comprises the disposition of disposable markers at different locations on the exterior of the portion of the body being treated and that is subject to movement during surgery. For example, such a body portion can be the chest cavity or the lower abdominal area, or the cranium. The spatial locations and orientations of these respective markers are each tracked with accuracy in relation to a predesignated three-dimensional coordinate system that is fixed in space, such as the operating room. If the surface being tracked has been mapped during breathing, such as breathing with or without anesthesia, prior to the time of treatment with high-energy radiation, high energy ultra sound radiation, or a remote controlled scalpel, that prior mapping can be integrated into the predetermined path to be followed by the scalpel (solid or radiation). Further, the prior tracked breathing movements can be compared to breathing movements being tracked during surgery and any differences superimposed on the predetermined movement tracking input to the surgical path. Such pre-operative tracking of the patient's movements may be used to determine a tracking algorithm that can be used by a tracking or robotic surgical system during surgery to determine the instantaneous location of the target (e.g., a tumor being irradiated) as a result of and during patient movements (e.g., breathing, coughing, etc.) so that the radiation source can remained focused on the target during such movements. In this manner, the direction as well as the power of the high energy radiation scalpel, or of the remotely controlled solid or radiation scalpel, or the like, may be continually adjusted based on the instantaneous positions and orientations of the external markers so that the diseased tissue that is sought to be resected or rendered necrotic is substantially the only target to the greatest extent possible. Adjustments to the direction of a solid or radiation scalpel may be accomplished using any available controllable degree of freedom in the surgical system, including movement of the scalpel or radiation source (e.g., via robot movements), redirecting the radiation beam at the collimator (e.g., via adjustments to collimator focusing elements), and/or movement of the patient (e.g., by moving the operating room table, bench or couch on which the patient is positioned).

There exists certain commercially available software, for example from Boulder Innovation Group, Inc., of Boulder, Colo., that is capable of performing these tracking activities both before and during surgery. The software used to track patient movements is, of course, and integral part of the instant invention. This invention would not be operative without such software as part of the instant invention, and the software is used in combination with disposable supports for the radiation emitters and in combination with certain optical fibers and certain emitters, in the instant method.

In order to map the motion of a surface, including a constantly moving surface such as a chest cavity that is moving because of breathing, it is necessary to provide a tracker systems (referred to herein as a “receiver system” and “receiver means”) which can detect and track the locations of markers placed on the patient, such as by receiving and tracking light or other radiation emitted from the markers. In an embodiment, the markers include a light emitting diode (LED). The tracker system is configured to receive the emissions from LEDs or the like and use the results of such reception to determine the locations and direction vectors of the emitters within a coordinate reference system (e.g., with respect to the operating room, the radiation source, etc.). Where the marker emissions are radiation in the electromagnetic spectrum wavelengths (e.g., visible light), it is common to use as a receiver a three (3)-camera array to enable accurate location calculation of the transmitted information in three dimensions. While three-camera arrays are commonly used with LEDs emitters, two-camera arrays, as well as a one-camera array system may be used.

A receiver system is configured so that the receivers (e.g., cameras) are disposed within line of sight of the markers so that emissions from the emitters on the markers can be picked up by the receivers and thereby are adapted to be converted into positions and direction vectors of the markers. The positions and direction vectors of the markers as so determined can then converted into a map of the moving outside of the body being worked on. When the marker emitters emit electromagnetic radiation, the receiver system may comprise one or more cameras that work together to determine the current locations of the marker emitters so that a constantly changing map of markers can be established.

In one aspect of this invention, the means by which the marker is attached to the outside of the body is disposable. By using disposable markers, the risk of transmitting infections and the like can be substantially reduced, if not eliminated entirely. However, under certain circumstances, it is considered to be within the scope of this invention for the means by which the markers are attached to the outside body surface(s) to be reusable. Although the preferred commercial embodiment of this invention may employ disposable markers, disposability is not an indispensable feature of all aspects of this invention.

Although a single marker may be used in the instant invention, it is preferred to use a plurality of markers. The markers that are disposed on the patient's body are configured to emit radiation, such as an LED (preferably emitting light in the visible red and invisible infrared wavelengths), which can be received by receivers in the tracking system. Alternatively, or in combination, a receiver device that responds to a magnetic field, or a reflector of light (visible or not), or an emitter of visible light, or a laser diode, or ultrasound, or their equivalents, can be used. Collectively the various markers are sometimes hereinafter referred to as “emitters” and “signal emitters” since the emitted or reflected radiation provides a signal that can be recognized and processed by a receiver device. Nothing contained herein, however, should be construed as limiting the words “emitter” and “signal emitter” to LEDs or the like unless specifically recited in the claims. Rather, emitters and signal emitters should be construed to mean any device capable of indicating the location and direction vector of a marker relative to a tracker system or controller. As used herein, the term “signal” is intended to encompass whatever emitted radiation is employed to communicate between the marker and the tracker system or controller, such as for example, emitted radiation, reflection radiation, disruption of a magnetic field, ultrasound, etc.

Emitter markers that emit—rather than merely reflect—radiation may require a power source in order to enable them to emit radiation. Thus, the emitter marker may have battery power attached thereto, or be configured to be attached to an outside source of electric power. An element that produces a magnetic signal can be powered by an electromagnet, which may have access to a source of power, such as a battery or external line current, or by a permanent magnet, which does not need a power source. The power connector may be attached directly to the marker in need of a power source.

A reflecting emitter, of course, does not require electric power input. A reflecting emitter merely requires a reflective material or structure (e.g., a corner cube structure) disposed on an exposed surface configured to reflect radiation (e.g., visible light or UV light) shining on the emitter so that reflected radiation received by a suitable receiver can be used to determine the position and orientation of the marker.

In an alternative embodiment, the markers can be electromagnetic sensors that respond to an externally generated and applied radiation or magnetic field.

Other similar radiating markers are well known, per se, and will be apparent to those of ordinary skill in this art. The above referred to markers are merely exemplary.

Where external power is required, the marker includes a structure for electrically connecting to a suitable power source (e.g., internal battery or a connector for connecting to an external power source). For example, a conventional connector or clip can be used. The marker may be directly attached to an element, preferably a disposable element, that can be adhered, in a relatively fixed location and orientation, to the patient's skin. The marker/support element combination is positioned on the patient so that the transmissions therefrom can be accurately received by a receiver of the tracking system. From the direction; and possibly the strength, of the received radiation, the tracking system can calculate the location and direction vector of the emitter/marker. By very accurately tracking a plurality of marker emitters, the tracker system can determine the motion of the surface supporting the emitters (the chest for example), such as movement that results from the patient's breathing. By tracking the emissions of the markers over short time intervals, the tracker system can track and map movement of the surface as it is occurring. This information can then be integrated with the preprogrammed the path of the surgeonless scalpel/high energy radiation, as may be controlled by a surgical robot, the radiation source and/or the operating table on which the patient is positioned on a substantially instantaneous basis, thereby substantially constantly adjusting the surgical path to account for the motion of the surface.

In a preferred embodiment, the emitter is an LED that emits radiation in the visible red or invisible infrared region of the spectrum. There are two alternative embodiments using such LED emitters. In a first embodiment, the LEDs are attached directly to the skin of the patient and are disposed at an angle such that their transmitted radiation can be directed to a camera array, comprising at least one camera that is adapted to receive such transmissions and that is mounted in a fixed location. The LEDs can be fired (i.e., illuminated) in a predetermined sequence so that the calculating software knows which LED has fired at any specific time, and therefore which location on the patient's body is being tracked. Alternatively, the various LEDs can be selected or configured to emit radiation of different wavelengths, which also enables discriminating between the various marker emitters. If different wavelength emissions are used, all of the emitters may be fired simultaneously or continuously because discrimination is a function of the wavelengths being emitted. Alternatively, LEDs can be fired simultaneously, using the same wavelength, provided that software that differentiates between simultaneously firing emitters is employed.

In this embodiment, LEDs that are attached to the patient's skin may be operatively associated with a power source. The power source can be a battery, but more preferably will be a line current, which means stringing an electrically conducting wire across the patient. This causes two potential problems that must be taken into consideration by the operator. First, there is the danger of patient leakage current exceeding that specified by medical regulatory bodies for contact with the electrical conductors. The second is the fact that such conductive wires are quite opaque to most radiation. Therefore, the wires themselves can interfere with the accuracy of the operation by attenuating the high energy treating radiation that is being impinged on the patient.

In an alternative embodiment, the LED(s) and their power supply are positioned away from the patient and optical fibers lead from the LEDs to positions on the skin of the patient where light is emitted into the operating room. As these remote LEDs fire, the light is transmitted through the optical fibers to a location on the skin of the patient where the fiber ends and from which light is projected toward the camera array of the tracker system. The advantages of this embodiment are twofold. First, no electrical wiring is in contact with the patient thereby eliminating the risk of exceeding patient electrical isolation requirements. Second, in applications in which the scalpelless surgical tool is high energy X-radiation, an LED can be selected such that it can cooperate with optical fibers that are more transparent to the high energy radiation. While all optical fibers are more transparent to high-energy radiation than are metal electrical wires, plastic optical fibers, especially methyl methacrylate fibers, attenuate infrared light transmitted through it more than they attenuate visible light. In the preferred embodiment, plastic optical fibers are preferably used with visible red LEDs or other visible light sources, and glass optical fibers are preferably used with infrared LEDs or laser diodes. Glass fibers are more opaque to high energy radiation than is plastic fibers.

In some operations, it has been found to be efficient to employ a combination of mensuration techniques, both as a double check and in order to insure that all movements are accurately determined. For example, markers could use a combination of LED electromagnetic radiation emitters and magnetic field generators (i.e., magnets). Where optical fibers are used to transmit the electromagnetic radiation from a remote source into energy being beamed to a camera, the optical fibers will not interfere with the magnetic field emitted by markers.

The marker element supporting the emitter, reflector, or the like, may itself be made of a material that reflects the movement of the surface that it is attached to. The support material may be such that tracking the movements of the emitter(s), or the like, necessarily tracks the supporting element and, through the supporting element, tracks the movement of the spot on the surface to which the supporting element/emitter(s) is attached.

While the element attaching the emitter to the moving surface being tracked should be configured to track the movement of the surface substantially identically, the marker is also preferably inexpensive because it is preferably disposable. Generally speaking, a paper or cardboard supporting element will not serve in this application because, although these materials are inexpensive, they are also is not particularly stable. In these cases, the movement of the surface to which they are attached may cause different portions of these supporting elements to move in a non-linear manner with respect to the surface. This may cause unacceptable variations in the tracking results and may cause inaccuracies in what is intended to be a very accurate tracking of a respectable structure. Further, body effluent, such as sweat, may cause deterioration of paper or cardboard elements that are exposed to it and it may even deteriorate certain kinds of cloth. On the other hand, paper or cardboard coated with a non-absorbent plastic, or the like, and that has been rendered adherent to the skin of the patient, may be well suited to use in this invention. Alternatively, supports that are made entirely of non-absorbent plastic elements, and that are adherent to the patient's skin, can be used in this invention.

In some instances, the supporting element should be relatively rigidly adhered to the underlying surface (skin) of the patient so that it will move directly with the movement of the underlying tissue that it is disposed on. However, it has been found that in some cases, a flexible material will serve very well as the supporting structure. Thus, for example, a textile fabric or a plastic film, that are suitably not adversely affected by body effluent (e.g. sweat) can be used in this service under certain conditions.

If a fabric or film is stretched to conform to a body part surface whose movements need to be tracked, it can be adhered to the surface of the body part or not, provided that it substantially continually conforms to the surface of the body part and that it continues to so conform as the patient breathes. The key property of the support element is that it accurately translates the movements of the body part to the emitter so that the emitter can transfer these exact movements to the control means that integrates these movements with the preprogrammed surgical route to form a continually changing modified surgical route, and directs the movements of the scalpel or radiation in consequence of this modified surgical route.

In a preferred aspect of this invention, where the emitter is electrically powered and is itself disposed on the surface that is subject to movement, the supporting element, whether rigid or film form, should be substantially insulating, so that the electric current that is input to cause the emitter to operate will not cause patient discomfort. Therefore, metal support elements should be used sparingly and with great care. This objection to the use of metal support elements presents a problem where the marker's location and orientation are determined as a function of a magnetic field and the magnetic elements must be magnetizable metal. In this respect, it has been found to be desirable to provide a support element that has a magnetic metal armature and an insulating cover at least over that part of the element that will come into contact with the patient's skin. Various commercially available, or yet to be invented, relatively rigid plastic materials will serve well in this function. In the case of a radiation emitter, such as a visible red, or an invisible infra red LED, the entire substantially rigid support element, or the film/fabric supporting layer, can be made of a non-conductive plastic or textile material or it may be made of metal carrying and insulating coating of insulating plastic.

In a preferred embodiment of this invention, the camera array, or other receiver, is suitably located in a fixed position, such as on the ceiling of an operating room. This position gives the greatest interference free view of the patient and the emitters that are attached to the patient. The LEDs or other emitters, including the radiating ends of the optical fibers, if that embodiment is employed, are suitably attached to a base member, which may be wedge shaped so that their radiation is principally directed toward the ceiling and especially toward the camera array mounted on the ceiling. Where the camera array is mounted such that it views the patient at an angle of approximately 45 degrees, the LED supports may be configured to cause the LEDs to emit radiation toward the camera array at a similar angle of approximately 45 degrees. Other spatial arrangements will be apparent to those of ordinary skill in this art.

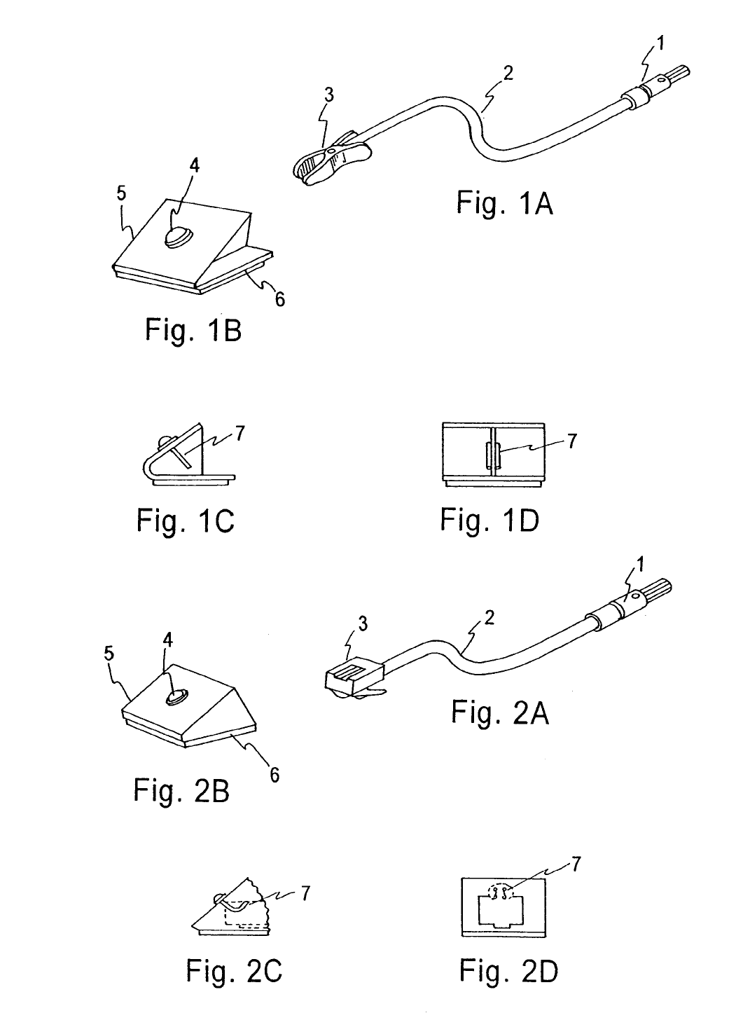

Referring to FIGS. 1-3, a cable 2 is adapted to be attached, at one end 1, to a source of external power (not shown) and, at the other end, to a plurality of suitable, preferably disposable, emitters 4. The emitters are shown as being attached to a supporting element 5 having one side that is suitably equipped with an adhesive material 6 that is adapted to adhere to a patient (not shown).

FIG. 1A is a schematic representation of one aspect of this invention that shows the cable 2 with a suitable connection, that is adapted to connect 1 to an external power supply (not shown) and a clip 3 that is adapted to connect to a lead 7 from an LED. FIG. 1B is a schematic representation of the same aspect of this invention where there is shown an LED emitter 4 and a support element 5 with an adhesive backing 6. FIG. 1C is a schematic representation of the same aspect of this invention that shows a side view of an emitter supporting element 5 and shows a lead 7 from the LED that is adapted to be attached to the clip 3. FIG. 1D is a schematic representation of this same aspect of this invention that is similar to FIG. 1C but shows an emitter with two leads 7 extending therefrom.

FIG. 2A is a schematic representation of another aspect of this invention that shows the cable 2 with a suitable connection 1, that is adapted to connect 1 to a power supply (not shown) and a connector 3 that is adapted to connect to a lead 7 from an LED. FIG. 2B is a schematic representation of this aspect of this invention where there is shown an LED emitter 4 and a support element 5 with an adhesive backing 6. FIG. 2C is a schematic representation of this aspect of this invention that shows a side view of an emitter supporting element 5 and shows a lead 7 from the LED that is adapted to be attached to the connector 3a. FIG. 2D is a schematic representation of this aspect of this invention that is similar to FIG. 2C but shows an emitter with two leads 7 extending therefrom for attachment to the connector 3a.

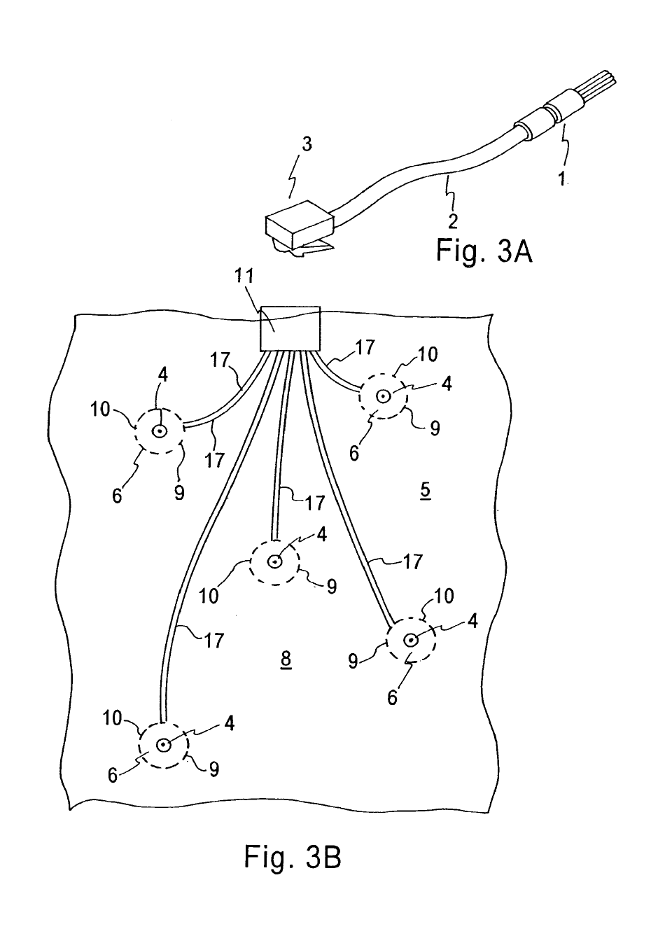

FIGS. 3A and 3B are schematic representations of a different aspect of this invention that employs a disposable fabric backing 8 to which multiple LED's 4 are attached. Each LED 4 is attached to an area 9 of the fabric under which a self-adhesive pad 10 can be disposed. It is considered to be within the scope of this invention that suitable adhesive material can be applied directly to the underlying fabric backing and to thereby enable the backing to be adhered to the body being worked on. The fabric may have suitable wiring 17 disposed on its surface and preferably attached to the fabric, or the wiring may be directly integrated into the fabric. The wiring 17 connects the several LEDs to a hub 11 that is adapted to be connected to a connector 3 that is in turn connected to a power supply (not shown) though a single or multiple electrical lead 2.

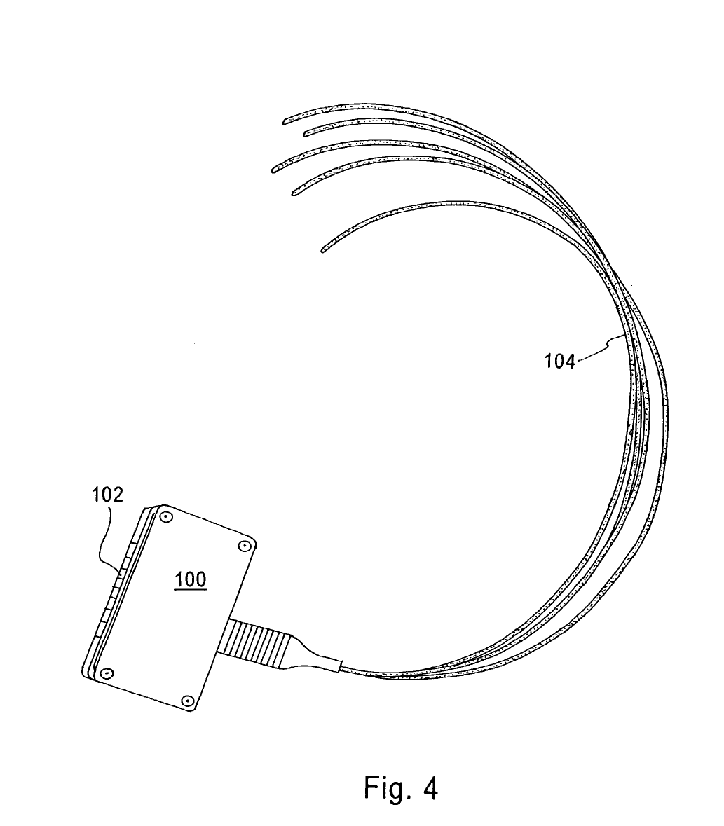

FIG. 4, shows a housing 100 in which is disposed at least one, but preferably a plurality of, LEDs (not specifically shown). Electrical connectors 102 are provided to supply electric power to the LEDs. In an embodiment, a tracking system may be connected to the electrical connectors and configured to supply power to specific electrical connectors 102 to control the firing of specific LEDs within the housing 100. In an embodiment, a timing device (not shown) may be provided operatively attached to the plurality of LEDs to cause the LEDs to fire in a preprogrammed sequence. A plurality of optical fibers 104, shown emanating from the housing 100, that are suitable for attachment to markers that are in turn suitable for attachment to a body.

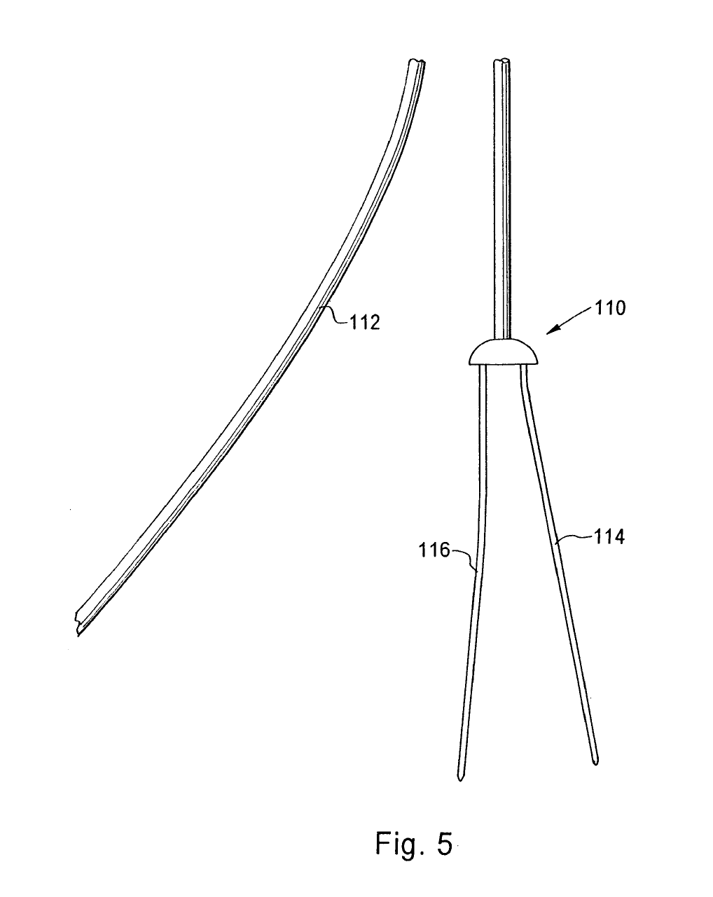

FIG. 5 shows an emitting LED 110 that is attached to an optical fiber 112. The LED has two leads 114 and 116 that are attachable to a source of electric power (not shown). When the LED 110 fires, its light emission is captured by the optical fiber 112 and transmitted through the fiber to its terminus located at a position on the outside of the patient's body being treated (not shown) from which location the light is projected from the end of the optical fiber toward a camera array (not shown) where the movement of the skin of the patient is tracked.

As described above, adjustments for patient movements may be accomplished by moving the operating room table on which the patient is positioned. This embodiment system is used in substantially the same way as the robotic surgery embodiments described above, except that instead of sending positioning signals to control the robot, the positioning signals are sent to a computer-controlled bed or operating table 618 on which the patient is lying. In this embodiment, the bed moves in directions to compensate for the patient's breathing or other movements with respect to an external frame of reference (e.g., the operating room) so that the target (e.g., a tumor) remains still relative to the radiation source or other surgical instrument. In this embodiment, the motion of the target is tracked and compensated for so that the bed or table will move in directions opposite that of the target (e.g., tumor), as it may be moved by patient movements. As mentioned above, movements of the target may be calculated using a correlation algorithm derived from the patient's breathing, as well as knowledge of tumor movement relative to said patient's breathing, as may be obtained during pre-operation imaging. For example, the correlation algorithm may be derived by imaging the patient while monitoring the patient's movements, such as breathing, using the same or similar tracking system, with markers positioned on the same locations on the patient as during surgery. In this manner, the position of the target obtained by the imaging can be correlated to the positions and movements of the patent over time and within observed ranges of movement (i.e., the movements of the patient that occurred during imaging).

Also as described above, adjustments for patient movements may be accomplished by moving the radiation source collimator. Modern radiation sources rely on collimators to shape and restrict the radiation beam to the required size and form. These high resolution collimators are commonly of multi-leaf designs that offer a dynamically modulated aperture similar to a camera aperture except that the opening may be repositioned and shaped in addition to be restricted in diameter. Thus, collimators can “focus” radiation from a source into a “beam” by blocking or shading the edges of the beam, attenuating the radiation in all directions except through the opening in the collimator. By controlling the leaves of the collimator in a side-to-side fashion (instead of just open and close) the beam can be made to “move” laterally with respect to the long axis of the collimator. Such movements by adjusting the collimator leafs enables the beam to be rapidly re-directed with fine tolerances compared to the course aiming of the beam that can be achieved by reorienting the radiation source and collimator assembly, such as by means of its robotic support system. Some collimator systems may also include focusing elements or “mirrors,” such as heavy metal (e.g., gold, tungsten or iridium) sleeve or tube oriented along the long axis of the collimator which can scatter radiation through a small grazing angle (e.g., 3.72 degrees for gold mirrors) so as to further focus the radiation exiting the collimator. In the future, such focusing elements may also be controllable in order to enable the focused beam to be further steered. By linking such collimator control mechanisms to measured movements of the patent, and more particularly to movements of the target (e.g., tumor) as determined from measured movements of the patient, the collimator can cause the radiation beam to follow and, thus remain focused on, a moving target, such as a target tumor. In particular, a computer of a the surgical system may be configured with computer-executable instructions to control the collimator elements to cause the radiation beam exiting the collimator to move so as to compensate for movement of the patient's body during treatment of the inside portion of the body. Such collimator movement commands may cause the controllable radiation blocking elements (e.g., leafs) to adjust the portion of the radiation that is attenuated by the blocking elements so that the beam portion of the radiation exiting the collimator is effectively steered so as to compensate for the patient movements so that the radiation energy remains focused on the target (e.g., a tumor).

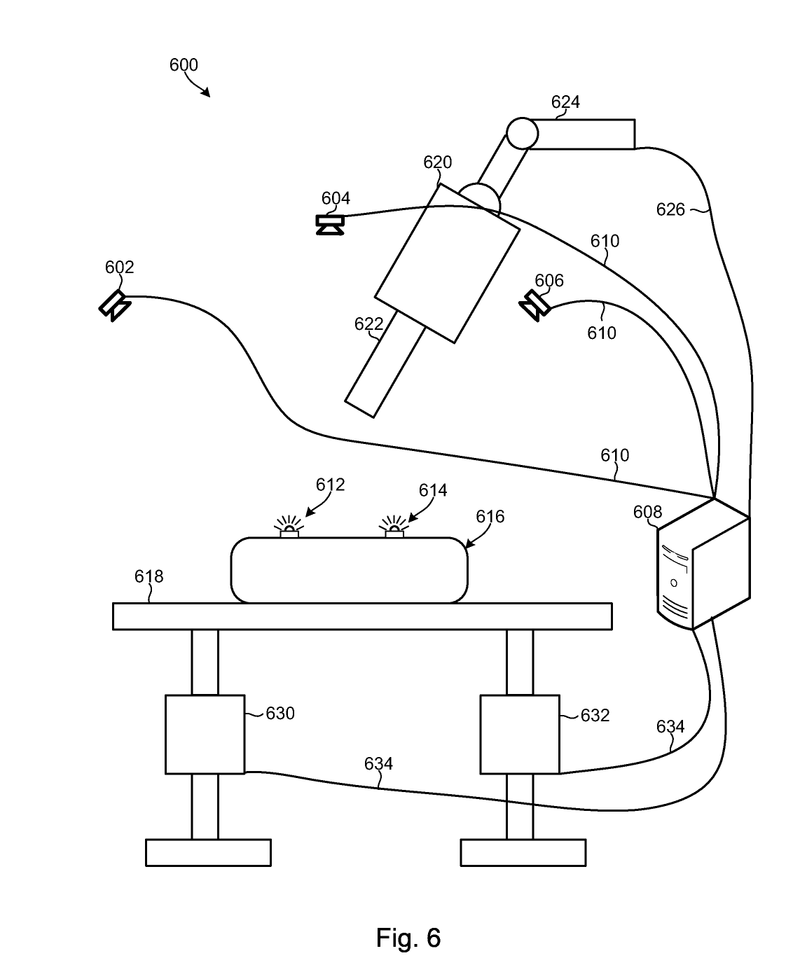

FIG. 6 illustrates the various components of the tracking system marker emitters and surgical system described above. The various components may be integrated into a precision surgical system 600 that includes a tracking system 602-608, a robotic surgical system 624, a positionable operating table 618, and a plurality of markers 612, 614 positioned the patient 616. As described above, light or other emissions from markers 612, 614 positioned on a portion of the patient 616 are received by receiver devices 602, 604, 606, such as digital cameras. Signals from the receiver devices 602, 604, 606 may be conveyed to a tracking system computer 608 (also referred to as a “controller”), such as by cables 610 or wireless data links (not shown). As discussed above, the information provided by receiver devices 602, 604, 606 may be processed by the tracking system computer 608 using known triangulation or similar algorithms to determine the positions of each light emitting marker 612, 614 within an external coordinate system, such as a coordinate system linked to the robotic surgery system 624 or the operating room.

In a particularly useful application, the robotic surgery system may comprise a high-energy radiation source 620, such as a high-energy X-ray generator or radioisotope chamber, that is coupled to a collimator 622. As is well known, high energy radiation source collimators 622 may be configured with beam forming elements (e.g., shutters, beam guides, movable shielding, etc.) that are configured to generate a very narrow beam of radiation. Some configurations of collimators 622 may include movable elements that can be manipulated by a controller in order to precisely steer a highly collimated beam of radiation. In order to precisely aim the beam of radiation at a target within the patient, the radiation source 620 and collimator 622 may be coupled to a computer of the treatment system or controlled by a robot system 624. In an embodiment, the collimator 622 and/or robot system 624 may receive aiming and fine adjustment movement commands from a controller, such as the tracking system computer 608 via a control cable 626. As described above, positioning commands from the tracking computer 608 may be issued to the collimator 622 and/or surgical robot 624 to compensate for movement of the patient 616 detected by the tracking system based on the determined locations of the markers 612, 614. In this manner, a highly collimated beam of radiation emanating from the collimator 622 may remain focused on the target (e.g., a tumor) even as the patient 616 moves and breathes.

Also as mentioned above, patient movements may be compensated for by adjusting the position of the patient 616 by moving the operating table 618 on which the patient is positioned. For example, positioning commands from the tracking computer 608 may be issued to electrical or hydraulic positioners 630, 632 coupled to the operating table 618. Such electrical or hydraulic positioners 630, 632 may be configured to move the operating table 618 to adjust the position of the target as determined by the tracking system. For example, if the tracking system determines that breathing movements of the patient 616 are causing the target to move up and down in a rhythmic manner, such movement of the target may be compensated for by the tracking system computer 608 issuing movement commands to the operating table 618 actuators 630, 632 to cause the table to move down and up in a compensating manner. Thus, if the tracking system determines that the target has moved up by a half centimeter, the tracking system computer 608 may command the table actuators 630, 632 to move the patient 616 down by a half centimeter.

Further, as mentioned above, the tracking system computer 608 may issue movement commands to all of the controllable elements, including the collimator 622, surgical robot 624 and operating table actuator 630, 632, in a coordinated fashion in order to ensure that the beam of radiation remains fixed on the target regardless of the patient's movements. The controllable elements that are given particular movement commands may depend upon each element's speed of response, range of motion and positional accuracy. Thus, the surgical system may detect rhythmic up and down movements from the patient's breathing and compensate for such movements of the target by down and up movements of the operating table 618, while side to side movements are addressed by fine adjustments to the beam controlling components of the collimator 622, and large adjustments by the surgical robot 624.

While FIG. 6 shows the tracking system computer 608 controlling each of the movable elements of the surgical system 600, it will be appreciated by one of skill in the art that multiple processors may be combined or linked to function as a coordinated system. Thus, the controller 608 of the tracking system may pass target location and movement information to one or more controllers associated with the movable elements or an overall control processor (not shown separately).

In general, the operation of the three embodiments described above (i.e., surgical robot controls, robotic table, and computer-controlled collimator) are functionally similar using the same basic patient tracking system, with the fundamental difference being which device accommodates movements of the target. Also as described above, the robotic operating room table 618 and the surgical robot 624 or the computer-controlled collimator 622 may be controlled simultaneously. For example, the system may compensate for large movements of the target, such as in response to a cough or muscle movement, by moving the operating room table while small movements, such as in response to breathing and heart beats, may be compensated for by moving the robot or the collimator.

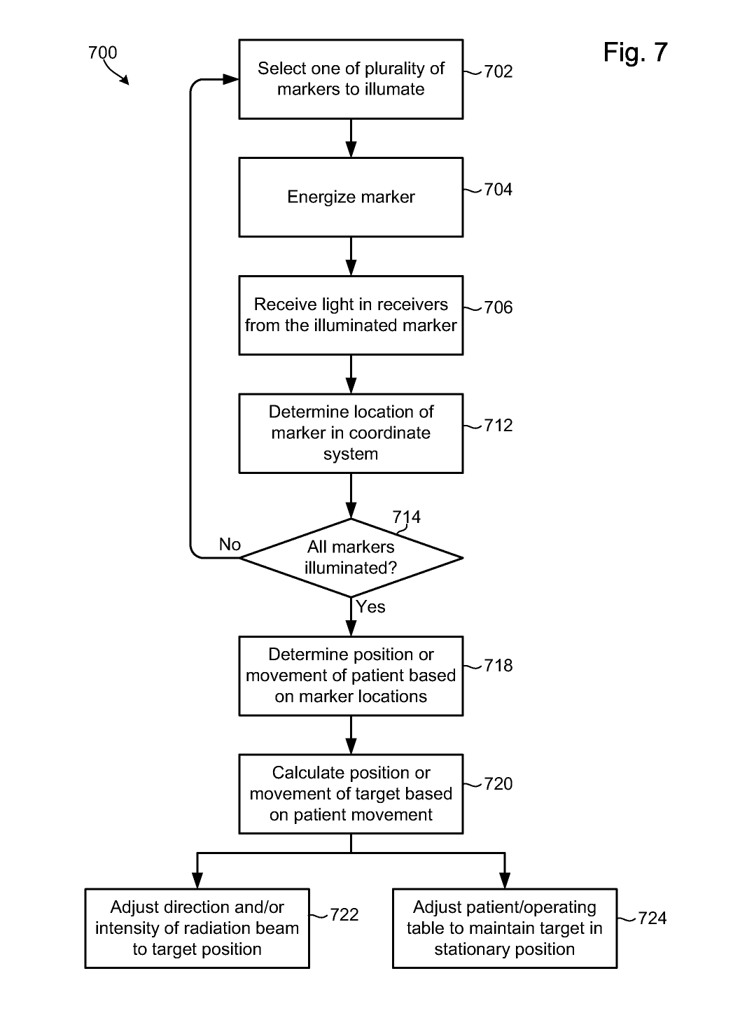

An example method 700 for controlling a surgeonless surgical system as described above is illustrated in FIG. 7. In an implementation in which the emitters on a plurality of markers are sequentially illuminated under control of the tracking system, the tracking system computer 608 may select one of the markers to illuminate in step 702. In step 704, the selected marker is illuminated, and in step 706, the receiver devices of the tracking system receive the light (or other radiation) emitted by or reflected from the emitter on the illuminated marker, and generate positioning information that is sent to the tracking system computer 608. In step 712, the tracking system computer 608 processes the generated positioning information using well known triangulation algorithms to locate the illuminated marker within a coordinate system. As mentioned above, this coordinate system may be an external frame of reference, such as 3-D coordinates related to the operating room or the surgical system (e.g., high-energy radiation emitter and collimator).

The positioning information generated by the receiver devices in step 706 and used by the computer 608 to determine the marker location in step 712 is information that the receiver device generates based upon or in response to the received emitted radiation. Since the type of positioning information generated by the receiver device will depend upon the type of the emitted radiation and the type of receiver device (e.g., digital cameras, light detectors, microphones, magnetometers, radio frequency receivers, etc.), the general term “positioning information” is used herein to refer to the signal or data generated by receiver devices and provided to the tracking system computer 608. In embodiments in which visual or infrared light radiation is emitted from marker inventors, the receiver devices may be digital cameras, so the generated positioning information may be the pixels or locations within the digital image that detect the emitted light. When the same emitter is imaged by three or more digital cameras, the respective image pixel information generated by each of the cameras can be used by the tracking system computer to locate the emitter within a 3-D coordinate system using well-known trigonometric calculations. In embodiments in which the emitted radiation is light, radio waves or ultrasound, the receiver devices may be light detectors, radio receivers or microphones which record the time of arrival of the received radiation, so generated positioning information in such systems may be the precise time the signal was received. In such an embodiment, the time of arrival positioning information may be used by the tracking system computer 608 to determine the location of the emitter using well-known spherical triangulation calculations (e.g., similar to those used to locate earthquakes based upon seismic data). Such time of arrival information may be compared to the time of arrival in one detector, and/or to the time of an triggering signal, such as a light pulse emitted by the tracking system that is reflected from markers. In the case of RFID emitters as in the embodiment described below, such time of arrival information may take into account the lag time associated with the RFID chip processing and replying to a query signal transmitted by the tracking system. In embodiments in which the markers generate a magnetic field that is sensed by the tracking system, the generated positioning information may be information related to the magnetic field strength detected at the receiver device, which may be a magnetometer or similar a field sensor. Since a magnetic field strength may indicate a distance from the receiver device to the each marker, the embodiments utilizing a magnetic field emitter may also use spherical triangulation calculation to determine the location of markers.

In step 714, the tracking system computer 608 may determine whether all of the markers have been illuminated and located. If there are more markers to illuminate and track (i.e., determination step 714=“No”), the computer 608 may return to step 702 to select another marker for locating. Of course, if the marker emitters emit light in different wavelengths, enabling differentiation based on light wavelength (i.e., color), there may be no need to sequentially illuminate markers, in which case steps 702 and 714 are unnecessary and the location of all the markers may be accomplished in step 712 based upon location and light wavelength/color information provided by the receiver devices.

Once all markers have been located (i.e., determination step 714=“Yes”), the computer 608 may use the determined marker locations to determine the location or movement of the patient (e.g., movement since a last measurement or movement from a baseline position) in step 718. This may be accomplished by comparing marker locations in the present interval to locations in a previous interval and/or in a baseline or initial state. Using the determined patient position information, the computer 608 may then calculate the present position or movement the target volume (e.g., a tumor) within the patient in step 720. As mentioned above, this calculation may make use of measurements, models or algorithms developed during an imaging session during which the target was located within the body based on imaging (e.g., MRI, X-ray, ultrasound, etc.) accomplished while the patient's body location/position was monitored by the same or a similar tracking system. This calculation may alternatively make use of standard models or models created based upon imaging data.

Once the tracking system computer 608 has determined the current location or movement of the target within the patient, the system may send movement commands to one of the controllable elements within the surgical system 600 to compensate for the movement in order to keep the surgical tool (e.g., radiation beam) focused on the target. For example, in step 722, the computer 608 may send movement commands to the collimator 622 to redirect or refocus the high-energy radiation beam on the new location of the target. Alternatively or in addition, the computer 608 may send movement commands to a surgical robot 624 to reposition a surgical instrument (e.g., radiation source 720 and collimator) to align it with the new location of the target. As another alternative, in step 724, the computer 608 may send movement commands to actuators 630, 632 controlling the position of the operating room table 618 to maintain the target in the same location with respect to the radiation beam emitted from the radiation source 620 and collimator 622 or other surgical instrument.

The processes of method 700 may be accomplished continuously so that as quickly as the tracking system finishes one marker measuring process it starts over to perform the operations again. In this manner, the target's location and movement can be continuously monitored.

In a further embodiment, the tracking system may apply the same basic mechanisms used for tracking the patient to also track movable elements within the robotic surgical system 600, such as the operating table 618, the radiation source 620, 622 and/or the surgical robot 624. By doing so, the tracking system may be used in a closed-loop control process to finely control movable elements, such as movements accomplished by the operating room table actuators 630, 632. In this manner, the various embodiments may be used not only to track the location of a target, but also to control the position compensation movements of the system's movable elements in order to maintain the target in a fixed location within the 3-D coordinate system of the tracking system.

In a further embodiment, a memory chip or radiofrequency identifier (RFID) tag or chip may be included within each marker 712 and/or disposable harness 100 so that its identity and usage (e.g., number of uses, patent ID, etc.) can be recorded automatically by the tracking system. Information regarding the marker 712 and/or patient may be stored in the memory chip or RFID tag memory and transmitted to the tracking system as a signal encoded within a wired or wireless transmission.

RFID chips are well known and quite affordable. Adding an RFID chip to the markers and/or disposable harnesses can enable the tracker system to wirelessly query the RFID chip during a tracking session to obtain a static serial number. This reading of the RFID identification information may be accomplished when tracking happens with the ID recorded in the control software. Some RFID chips include read/write memory enabling them to record and report some data. In an embodiment, such RFID chips may be used and configured to provide a counter that is incremented each time the maker or harness is used. In a further embodiment, the RFID chip may include a static serial number and a read/write memory into which a count value as well as customer-specific information can be written to and read back from. While some RFID chips transmit radio frequency signals according to the RFID protocol, RFID chips may also transmit radio frequency signals according to other wireless communication protocols, such as Bluetooth, Near Field Communication protocol, Zigbee, IEEE 802.15.4, IEEE 802.11x, WiFi, WiMax, and cellular telephone protocols. Therefore, references to RFID tags and RFID chips in the various embodiments and the claims are not intended to limit the claims to particular wireless communication protocols.

Memory chips are also well known and quite affordable. A memory chip attached to or included in markers and/or a disposable harness may be configured to supply a signal encoded with identifier information when queried by a memory chip query device. Such a memory chip query device may be a circuit including a processor configured to submit a query message to the memory chip and receive the identifier encoded signal in reply. The communication with the memory chip may be via a wired (e.g., a data bus) or wireless data link. Such a wireless data link may be any known wireless data communication technology, including, for example, RFID, Bluetooth, Near Field Communication protocol, Zigbee, IEEE 802.15.4, IEEE 802.11x, WiFi, WiMax, and cellular telephone wireless data links. Such a memory chip query device may be configured to recognize or decode an identifier encoded in the signal emitted by the memory chip in response to a query signal, and to pass the identifier to other components of the tracking system. The tracking system may be configured to recognize the identifier encoded in each memory chip emitted signal and use this identifier as part of tracking patient movement (e.g., tracking movements of particular points on the patient's body).

In a further embodiment, an RFID chip embedded in the markers may serve as the emitter, emitting radiofrequency signals in response to a query signal transmitted by the tracking system. As is well known in the RFID arts, typical RFID chips emit a radiofrequency signal encoded with an ID number or other identifier in response to receiving a radiofrequency query signal. Some low cost RFID chips receive the power necessary to emit their response signal from the energy of the received query signal, and thus do not require a power source. In effect, such low cost RFID chips function similar to light reflecting materials except that they emit radiofrequency signals encoded with identifying information. Thus, in this embodiment, markers equipped with a low cost RFID chip can be applied to the patient. Since emitted radio waves will be emitted approximately spherically, this embodiment does not require positioning the emitter at an angle in order for the emissions to be received by tracking system receiver devices. The receiver devices may be simple radio frequency antennas tuned to the frequencies emitted by the RFID chips. The location of the markers may then be determined based upon the time of arrival of the response signals (e.g., in relation to the time the query signal was emitted). Since such low cost RFID chips are typically quite small and thin (as they are typically included within product labels and tags), the markers in this embodiment may be simple paper or plastic wafers or discs with an adhesive layer—the disposable support element—that can be applied directly to the patient's skin. Since RFID chips are typically configured to emit a unique code or ID, this information may be received and used by the tracking system to distinguish each of the markers. Thus, the markers may be queried and tracked simultaneously as in response to a single query signal transmission by the tracking system.

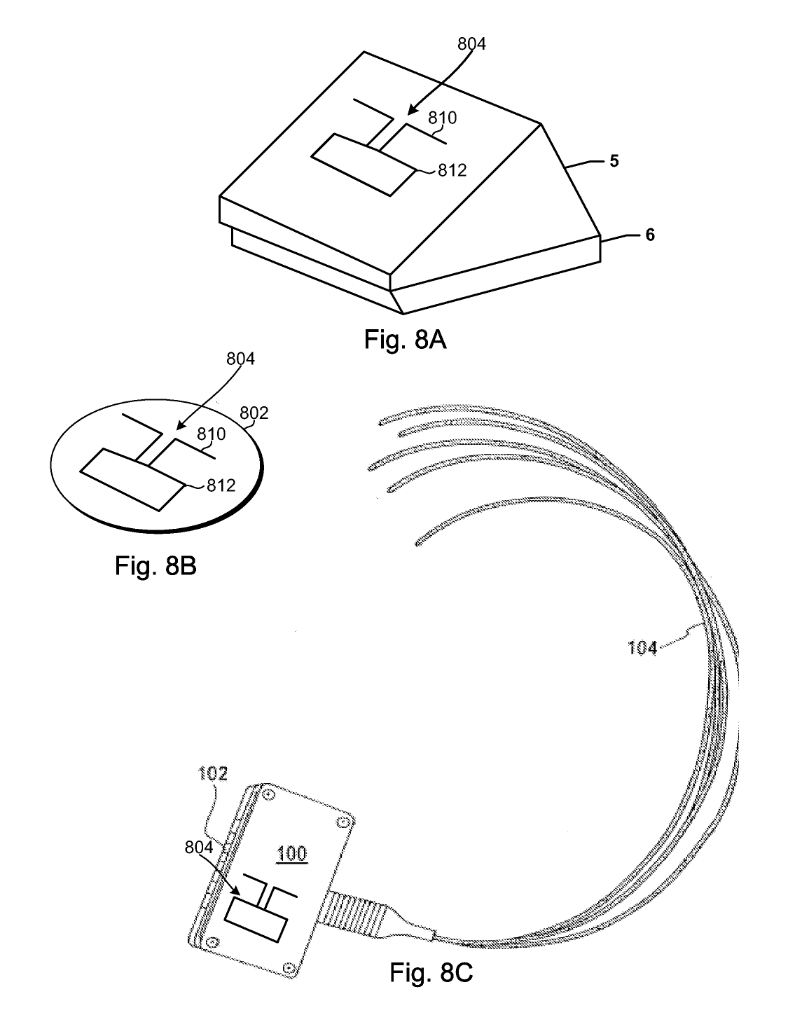

FIGS. 8A-8C illustrate three examples of including an RFID tag on markers or a disposable harness 100. FIG. 8A illustrates an embodiment in which an RFID tag 804 is positioned on a marker 5 in the form of a base member, which may be wedge shaped, coupled to a disposable support element 6. As is well known, and RFID tag typically includes a small antenna 810 electrically coupled to an integrated circuit 812. The antenna 810 receives radiofrequency energy from a reader which transmits a query signal, and transmits a radiofrequency response signal generated by the integrated circuit 812. The integrated circuit 812 typically includes a simple radio frequency receiver circuit, a circuit or memory element which encodes identifier information, and a simple radio frequency transmitter circuit coupled to the antenna 810. As mentioned above, due to the small size of RFID chips, they may be implemented within a small package, such as a thin disk 802 as illustrated in FIG. 8B. An RFID chip 804 may also be applied to a remote LED housing or disposable harness 100, as illustrated in FIG. 8C, to enable the tracking system to identify the particular harness or housing being employed in a measuring operation.

As discussed above, the integrated circuit 812 may be a memory chip that can be accessed by a memory chip interrogator via a wired (not shown) or wireless data link such as via the antenna 810. As mentioned above, the wireless data link implemented via the antenna 810 may be according any one of RFID, Bluetooth, Near Field Communication protocol, Zigbee, IEEE 802.15.4, IEEE 802.11x, WiFi, WiMax, and cellular telephone wireless data link protocols.

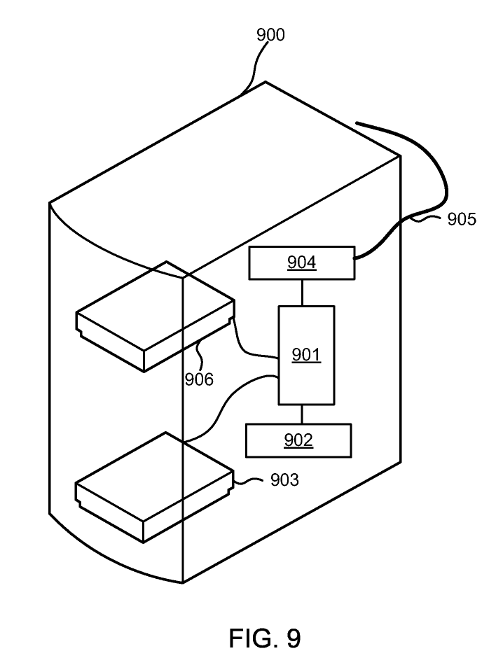

A number of the embodiments described above may also be implemented using a variety of commercially available computers, such as the computer 900 illustrated in FIG. 9. Such a server 900 typically includes a processor 901 coupled to volatile memory 902 and a large capacity nonvolatile memory, such as a disk drive 903. The server 900 may also include a floppy disc drive and/or a compact disc (CD) drive 906 coupled to the processor 901. The server 900 may also include network access ports 904 coupled to the processor 901 for establishing data connections with receiver devices and/or a network 905, such as a local area network for coupling to the receiver devices and controllable elements within a surgical system.

Computers and controllers used in the tracking system for implementing the operations and processes described above for the various embodiments may be configured with computer-executable software instructions to perform the described operations. Such computers may be any conventional general-purposes or special-purpose programmable computer, server or processor. Alternatively, some steps or methods may be performed by circuitry that is specific to a given function.

In one or more exemplary aspects, the functions described may be implemented in hardware, software, firmware, or any combination thereof. If implemented in software, the functions may be stored on or transmitted over as one or more instructions or code on a computer-readable medium. The steps of a method or algorithm disclosed herein may be embodied in a processor-executable software module which may reside on a tangible computer-readable storage medium. Computer-readable storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a machine readable medium and/or computer-readable medium, which may be incorporated into a computer program product.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A system for improving the accuracy of preprogrammed surgery on a body having an inside portion that is in need of surgery and an outside portion that may move during surgery, comprising:
treatment means for treating the inside portion of a body;
a plurality of markers each adapted to emit a corresponding plurality of signals and to be positioned on the outside portion of the body, each of the plurality of markers comprising:
a signal emitter configured to emit a detectable signal;
a base member coupled to the signal emitter; and
a disposable support element adapted to attach the marker to the body;
a tracking system configured to determine the location of the plurality of markers, the tracking system comprising:
one or more receiver devices configured to receive the signals emitted by the marker signal emitters and generate positioning information based upon the received signals; and
a computer configured to receive the information generated by the receiver devices; and
means for causing the marker signal emitters to emit signals under conditions sufficient to differentiate which emitter is sending each of said signals,
wherein the computer is configured with computer-executable instructions to perform operations comprising:
processing the generated positioning information received from the receiver devices to locate each of the markers within a coordinate system;
tracking movement of the outside portion of the body based on the processed generated positioning information;
identifying and mapping an inside portion of the body that is intended to be subjected to surgery based upon the tracked movement of the outside portion of the body; and
controlling the treatment means to compensate for movement of the body during treatment of the inside portion of the body,
wherein the combination of the emitter and the base member are adapted to dispose the emitter in line of sight with the one or more receiver devices.

2. The system of claim 1, wherein said treatment means comprises a radiation source and collimator configured to apply high energy radiation to the inside portion of the body along a predetermined path sufficient to render said inside portion of the body necrotic.

3. The system of claim 1, wherein said treatment means is adapted to be operated without benefit of a surgeon.

4. The system of claim 1, wherein:
the signal emitter comprises a light emitting diode; and
the one or more receiver devices comprise one or more imagers.

5. The system of claim 1, wherein:
the signal emitter comprises a light emitting diode; and
the one or more receiver devices comprises one or more digital cameras.

6. The system of claim 5, wherein means for causing the marker signal emitters to emit signals under conditions sufficient to differentiate which emitter is sending each of said signals comprises light emitting diodes of different light wavelengths.

7. The system of claim 5, wherein the markers further comprise a memory chip configured to supply a signal encoded with identifier information.

8. The system of claim 7, wherein the memory chip is coupled to a wired data link that is coupled to the computer, and wherein the computer is configured to obtain the identifier information from the encoded signal received from the memory chip via the wired data link.

9. The system of claim 8, further comprise a radiofrequency identifier (RFID) chip configured to emit a radiofrequency signal generated according to a wireless communication protocol selected from the group consisting of RFID, Bluetooth, Near Field Communication (NFC), Zigbee, IEEE 802.15.4, IEEE 802.11x, WiFi, WiMax, and cellular telephone protocols.

10. The system of claim 5, wherein the markers further comprise a radiofrequency identifier (RFID) chip configured to emit a radiofrequency signal encoded with identifier information and generated according to a wireless communication protocol selected from the group consisting of RFID, Bluetooth, Near Field Communication (NFC), Zigbee, IEEE 802.15.4, IEEE 802.11x, WiFi, WiMax, and cellular telephone protocols.

11. The system of claim 5, wherein means for causing the marker signal emitters to emit signals under conditions sufficient to differentiate which emitter is sending each of said signals comprises a power source coupled to each marker and to the computer which is further configured with computer-executable instructions to individually energize each signal emitter.

12. The system of claim 1, wherein:
the signal emitter comprises a memory chip;
means for causing the marker signal emitters to emit signals under conditions sufficient to differentiate which emitter is sending each of said signals comprises a memory chip query signal transceiver configured to query the memory chip and receive encoded signals from the memory chip; and
the tracking system is configured to recognize an identifier encoded in signals emitted by the memory chip emitted signal.

13. The system of claim 12, wherein the memory chip query signal transceiver is configured to query the memory chip and receive encoded signals from the memory chip via a wired data link.

14. The system of claim 12, wherein the memory chip query signal transceiver is configured to query the memory chip and receive encoded signals from the memory chip via a wireless data link.

15. The system of claim 1, wherein:
the signal emitter comprises a radio frequency identifier (RFID) chip; and
means for causing the marker signal emitters to emit signals under conditions sufficient to differentiate which emitter is sending each of said signals comprises an RFID query signal transmitter and the tracking system configured to recognize an identifier encoded in each RFID emitted signal.

16. The system of claim 1, wherein the signal emitter comprises a plurality of light emitting diodes (LEDs) disposed in a housing remote from the body, the housing coupled to at least one fiber optic cable having an end that is operatively associated with each of the LEDs within the housing and having another end that is adapted to be substantially fixedly disposed on the outside portion of the body.

17. The system of claim 16, wherein the housing further comprise a radiofrequency identifier (RFID) chip configured to emit a radiofrequency signal encoded with identifier information and generated according to a wireless communication protocol selected from the group consisting of RFID, Bluetooth, Near Field Communication (NFC), Zigbee, IEEE 802.15.4, IEEE 802.11x, WiFi, WiMax, and cellular telephone protocols.

18. The system of claim 16, wherein the housing further comprise a memory chip configured to transmit a signal encoded with identifier information to the tracking system via a wired data link.

19. The system of claim 18, wherein the memory chip is coupled to the tracking system via a wired data link and the memory chip is configured to transmit the signal encoded with identification information via the wired data link.

20. The system of claim 18, wherein the memory chip is coupled to a wireless transceiver configured to transmit the signal encoded with identifier information to the tracking system via a wireless data link using a wireless communication protocol selected from the group consisting of RFID, Bluetooth, Near Field Communication (NFC), Zigbee, IEEE 802.15.4, IEEE 802.11x, WiFi, WiMax, and cellular telephone protocols.

21. The system of claim 1, wherein said treatment means comprises an emitter of high energy ultra sound radiation.

22. The system of claim 1, further comprising an operating table comprising positioning mechanisms configured to move the operating table in response to movement commands received from the computer,
wherein the computer is configured with computer-executable instructions to perform operations comprising issuing movement commands to the operating table positioning mechanisms to compensate for movement of the body during treatment.

23. The system of claim 1, wherein:
the treatment means comprises:
a high energy radiation source; and
a collimator comprising radiation blocking elements configured to collimate high energy radiation emitted from the radiation source so as to apply radiation to an inside portion of the body; and
the computer is configured with computer-executable instructions to perform operations such that controlling the treatment means to compensate for movement of the body during treatment of the inside portion of the body comprises issuing movement commands to the collimator controllable radiation blocking elements to re-direct radiation exiting the collimator so as to compensate for movement of the body during treatment.

24. The system of claim 1, wherein:
the treatment means comprises:
a high energy radiation source; and
a collimator comprising controllable radiation focusing elements configured to focus radiation from the high energy radiation source into a beam suitable for radiating the inside portion of the body; and
the computer is configured with computer-executable instructions to perform operations such that controlling the treatment means to compensate for movement of the body during treatment of the inside portion of the body comprises issuing movement commands to the collimator controllable focusing elements to steer the radiation beam so as to compensate for movement of the body during treatment.

25. A method of treating an inside portion of a body that is in need of surgery that may move during surgery, comprising:
placing a plurality of markers on an outside portion of the body, each of the plurality of markers configured to emit a detectable signal so that it may be received by a plurality of receiver devices under conditions sufficient to differentiate which marker is sending each detectable signal;
receiving the emitted detectable signal from the plurality of markers;
determining a location of each of the plurality of markers based upon the received emitted detectable signals;
determining a location of the inside portion of the body based upon the determined locations of the plurality of markers; and
controlling a radiation treatment applied to the inside portion of the body to compensate for movement of the body during treatment by accomplishing one of adjusting a position of an operating table supporting the body, adjusting a collimator in a radiation source, and adjusting both a position of an operating table supporting the body and a collimator in a radiation source.

26. The method of claim 25, further comprising:
transmitting a radiofrequency identifier (RFID);
receiving an RFID response signal encoding identification information; and
identifying one of the plurality of markers based on the encoded identification information within the received RFID response signal.

27. The method of claim 25, further comprising:
querying a memory chip on one of the plurality of markers;
receiving an response signal from the memory chip encoding identification information; and
identifying one of the plurality of markers based on the encoded identification information within the response signal received from the memory chip.
